(19) United States
(12) Patent Application Publication
(10) Pub. No.: US 2017/0340450 A1
(43) Pub. Date: Nov. 30, 2017

(54) Reverse Ankle Replacement System

(71) Applicants: Jose Bernardo Toro Arbelaez, Remsenburg-Speonk, NY (US); Craig Eric Klinger, Great Neck, NY (US)

(72) Inventors: Jose B. Toro Arbelaez, Remsenburg-Speonk, NY (US); Craig E. Klinger, Great Neck, NY (US)

(21) Appl. No.: 15/600,771

(22) Filed: May 21, 2017

Related U.S. Application Data
(60) Provisional application No. 62/341,457, filed on May 25, 2016.
(60) Provisional application No. 62/350,669, filed on Jun. 15, 2016.

(51) Int. Cl. A61F 2/42; A61F 2/30

(52) U.S. Cl. CPC A61F 2/4202; A61F 2/30771; A61F 2/30767; A61F 2002/4207; A61F 2002/3093; A61F 2002/30621; A61F 2002/30574; A61F 2310/00005; A61F 2310/00011; A61F 2002/30772

(57) ABSTRACT

The presented invention is a system for performing reverse ankle replacement in the setting of ankle joint osteoarthritis, and other degenerative conditions of the ankle joint including avascular necrosis. The presented tibial and talar components can be implanted in a cement-less or cemented fashion. The prosthesis of the presented invention consists of (1) a tibial component which includes a planar undersurface with porous texture applied to its upper, non-bearing surfaces, (2) a talar component which includes a bearing surface which is concave, (3) a polyethylene component which includes a bearing undersurface which is convex, and (4) multiple talar component embodiments with or without flanges and/or screw holes for accommodation of bone screws for additional fixation and for permitting sub-talar joint fusion.

### CROSS-REFERENCE TO RELATED APPLICATIONS

The present application claims the benefit of U.S. Provisional Patent Application Ser. No. 62/341,457, filed May 25, 2016 and U.S. Provisional Patent Application Ser. No. 62/350,669, filed Jun. 15, 2016 by the present inventors, the entire contents of each of which is incorporated herein by reference thereto.

### FIELD OF THE INVENTION

The present invention relates to ankle arthroplasty.

### BACKGROUND INFORMATION

Implants may be used, e.g., when one or more human (or animal) body parts need replacing. For example, known ankle replacement implants may have been developed, to treat such problems as, e.g., degenerative conditions of the ankle joint including ankle osteoarthritis.

The present orthopedic implants relate generally to ankle arthroplasty and more specifically, to a reverse ankle arthroplasty system including prostheses which can be fitted to prepared and shaped tibia and talus, and implanted with or without the use of bone cement, as the undersurfaces are porous in nature. The reverse nature of the ankle arthroplasty system is specific to the prosthetic bearing surfaces. The native shape of the human talar dome is convex, as viewed in the sagittal plane. Ankle replacement systems of the prior art also typically include talar components with similar convex bearing surfaces, as present in the human talar dome. An example of such from the prior art is shown in U.S. Pat. No. 4,069,518, which illustrates a talar member with a convex top bearing surface which articulates with a tibial member which includes a concave bearing undersurface. Another example from the prior art is shown in U.S. Pat. No. 8,591,595, which features a lower, talar component with a convex bearing surface which articulates with an intermediate part with an undersurface that is concave, with the intermediate component situated below an upper, tibial component.

The bearing surfaces of the talar component and the associated mating articular surface of the polyethylene component of the present invention is the reversed from the native joint surface of the human ankle joint, whereby the talar component articular surface is concave and the polyethylene component inferior articular surface is convex. The talar component of the present disclosure may yield a biomechanical advantage as the axis of rotation within talar component is in close proximity to the bottom surface of talar component. This allows for a short distance for stress transmission from the axis of rotation of the polyurethane component within the talar component, to the bottom surface of the talar component. The resultant increased stability of the talar component in at its interface with the underlying bone can improve the capacity for osseous-integration. This increased stability of the talar component along the underlying bone can lead to reduced incidence of aseptic loosening following ankle replacement. Implantation options are also provided for allowing additional fixation of the talar component through use of screw fixation and for fusing the talar-calcaneal joint in the setting of advanced osteoarthritis. Additionally, scalable talar implant and fixation options allow for adaptation of fixation based on underlying bone quality, as factors including osteoporosis and avascular necrosis. These fixation options allow for individualizing surgical decision making based on localized bone quality. The reverse ankle replacement of the present disclosure also offers the advantage of inclusion of a wide planar porous undersurface area to promote bony ingrowth. In one or more example implementations of the present invention, the talar component has a wide flat undersurface, with or without presence of talar stems. This large undersurface allows a wide planar surface for bony ingrowth in the presence of a porous undersurface.

### SUMMARY

The reverse ankle arthroplasty system according to an example embodiment of the present invention includes a talar component articular surface which is concave and the associated polyethylene component inferior articular surface which is convex. By reversing the articular surface, the talar component and utilizes a concave surface which yields a biomechanical advantage. The axis of rotation within talar component is close to the bottom surface of talar component. This allows for a short distance for stress transmission from the axis of rotation of the polyurethane component within the talar component, to the bottom surface of the talar component. Additionally, in one or more example implementations of the present invention, the talar component has a wide flat undersurface, with or without presence of talar stems. This large undersurface allows a wide planar area for bony ingrowth in the presence of a porous undersurface. Thus, a reduction in aseptic loosening may be achieved through use of an enlarged planar surface area for osseous-integration as compared to talar components of ankle replacements of the prior art. Additionally, multiple embodiments of the present invention may allow for use of screw fixation into the talus for further stabilization of the talar component. This can improve fixation strength in the setting of compromised bone quality and for fusion of the talar-calcaneal joint and improve initial implant stability.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1A is a right view from medial of a human right lower extremity and ankle including a distal tibia, distal fibula, talus, and calcaneus according to one or more example implementations of the disclosure;

FIG. 1B is a back view illustrating a human right lower extremity and ankle including a distal tibia, distal fibula, talus, and calcaneus according to one or more example implementations of the disclosure;

FIG. 1C is a right view from medial of a human right lower extremity and ankle including a distal tibia, distal fibula, and talus, following an ankle replacement procedure according to a previous device including a talar component with a convex bearing surface and intermediate component with a concave bearing undersurface;

FIG. 1D is a right view of a human right lower extremity and ankle demonstrating tibia and talus bone osteotomies and preparation for placement of reverse ankle replacement prostheses according to one or more example implementations of the disclosure;

FIG. 1E is a back view of a human right lower extremity and ankle demonstrating tibia and talus bone osteotomies and preparation for placement of reverse ankle replacement prostheses according to one or more example implementations of the disclosure;

FIG. 2A is a right view illustrating a tibial component including a tibial stem and tibial articular segment, according to one or more example implementations of the disclosure;

FIG. 2B is a back view illustrating the tibial component including the tibial stem and tibial articular segment according to one or more example implementations of the disclosure;

FIG. 2C is a top view illustrating the tibial component including a tibial articular segment superior surface.

FIG. 2D is a bottom view illustrating the tibial component including a tibial articular segment inferior surface according to one or more example implementations of the disclosure;

FIG. 2E is a right view illustrating a polyethylene component including a polyethylene component recess according to one or more example implementations of the disclosure;

FIG. 2F is a back view illustrating the polyethylene component including the polyethylene component recess according to one or more example implementations of the disclosure;

FIG. 2G is a top view illustrating the polyethylene component including a polyethylene component superior articular surface according to one or more example implementations of the disclosure;

FIG. 2H is a bottom view the polyethylene component including a polyethylene component inferior articular surface according to one or more example implementations of the disclosure;

FIG. 2I is a right view illustrating a talar component including a talar articular ridge according to one or more example implementations of the disclosure;

FIG. 2J is a back view illustrating the talar component including the talar articular ridge and talar stem according to one or more example implementations of the disclosure;

FIG. 2K is a top view illustrating the talar component including the talar articular ridge according to one or more example implementations of the disclosure;

FIG. 2L is a bottom view illustrating the talar component including the talar stem according to one or more example implementations of the disclosure;

FIG. 2M is a right view from medial of a human right lower extremity and ankle following placement of a reverse ankle arthroplasty system including the tibial component, polyethylene component, and talar component according to one or more example implementations of the disclosure;

FIG. 2N is a back view of a human right lower extremity and ankle following placement of a reverse ankle arthroplasty system including the tibial component, polyethylene component, and talar component according to one or more example implementations of the disclosure;

FIG. 3A is a right view illustrating a talar component including a talar articular ridge according to one or more example implementations of the disclosure;

FIG. 3B is a back view illustrating the talar component including the talar articular ridge and two talar stems according to one or more example implementations of the disclosure;

FIG. 3C is a top view illustrating the talar component including the talar articular ridge and two talar stems according to one or more example implementations of the disclosure;

FIG. 3D is a bottom view illustrating the talar component including the two talar stems according to one or more example implementations of the disclosure;

FIG. 3E is a right view from medial of a human right lower extremity and ankle following placement of a reverse ankle replacement system including the tibial component, polyethylene component, and talar component according to one or more example implementations of the disclosure;

FIG. 3F is a back view of a human right lower extremity and ankle following placement of a reverse ankle replacement system including the tibial component, polyethylene component, and talar component according to one or more example implementations of the disclosure;

FIG. 4A is a right view illustrating a talar component with screw fixation holes including a talar articular ridge according to one or more example implementations of the disclosure;

FIG. 4B is a back view illustrating the talar component with screw fixation holes including the talar articular ridge and talar stem according to one or more example implementations of the disclosure;

FIG. 4C is a top view illustrating the talar component with screw fixation holes including the talar articular ridge according to one or more example implementations of the disclosure;

FIG. 4D is a bottom view illustrating the talar component with screw fixation holes including the talar stem according to one or more example implementations of the disclosure;

FIG. 4E is a right view from medial of a human right lower extremity and ankle following placement of a reverse ankle arthroplasty system including the tibial component, polyethylene component, and talar component with screw fixation holes including four screws placed for additional fixation into the talus according to one or more example implementations of the disclosure;

FIG. 4F is a back view of a human right lower extremity and ankle following placement of a reverse ankle arthroplasty system including the tibial component, polyethylene component, and talar component with screw fixation holes including four screws placed for additional fixation into the talus according to one or more example implementations of the disclosure;

FIG. 5A is a right view from medial of a human right lower extremity and ankle following placement of a reverse ankle arthroplasty system including the tibial component, polyethylene component, and talar component with screw fixation holes including screws placed for both additional fixation and fusion of the talar-calcaneal joint according to one or more example implementations of the disclosure;

FIG. 5B is a back view of a human right lower extremity and ankle following placement of a reverse ankle arthroplasty system including the tibial component, polyethylene component, and talar component with screw fixation holes including screws placed for both additional fixation and fusion of the talar-calcaneal joint according to one or more example implementations of the disclosure;

FIG. 6A is a right view of a human right lower extremity and ankle demonstrating tibia and talus bone osteotomies and preparation for placement of reverse ankle replacement prostheses based on a shortened talar component according to one or more example implementations of the disclosure;

FIG. 6B is a back view of a human right lower extremity and ankle demonstrating tibia and talus bone osteotomies and preparation for placement of reverse ankle replacement prostheses based on a shortened talar component according to one or more example implementations of the disclosure;

FIG. 6C is a right view illustrating a shortened talar component with screw fixation holes including a talar articular ridge according to one or more example implementations of the disclosure;

FIG. 6D is a back view illustrating the shortened talar component with screw fixation holes including the talar articular ridge and talar stem according to one or more example implementations of the disclosure;

FIG. 6E is a top view illustrating the shortened talar component with screw fixation holes including the talar articular ridge according to one or more example implementations of the disclosure;

FIG. 6F is a bottom view illustrating the shortened talar component with screw fixation holes including the talar stem according to one or more example implementations of the disclosure;

FIG. 6G is a right view from medial of a human right lower extremity and ankle following placement of a reverse ankle arthroplasty system including the tibial component, polyethylene component, and shortened talar component according to one or more example implementations of the disclosure;

FIG. 6H is a back view of a human right lower extremity and ankle following placement of a reverse ankle arthroplasty system including the tibial component, polyethylene component, and shortened talar component according to one or more example implementations of the disclosure;

FIG. 7A is a right view illustrating a talar component with screw flanges including a talar articular ridge and talar stems according to one or more example implementations of the disclosure;

FIG. 7B is a back view illustrating a talar component with screw flanges including a talar articular ridge and talar stems according to one or more example implementations of the disclosure;

FIG. 7C is a top view illustrating a talar component with screw flanges including a talar articular ridge and talar stems according to one or more example implementations of the disclosure;

FIG. 7D is a bottom view illustrating a talar component with screw flanges including a talar articular ridge and talar stems according to one or more example implementations of the disclosure;

FIG. 7E is a right view from medial of a human right lower extremity and ankle following placement of a reverse ankle arthroplasty system including the tibial component, polyethylene component, and talar component with screw flanges including a talar articular ridge and talar stems with screw fixation holes including three screws placed for additional fixation into the talus according to one or more example implementations of the disclosure;

FIG. 7F is a back view of a human right lower extremity and ankle following placement of a reverse ankle arthroplasty system including the tibial component, polyethylene component, and talar component with screw flanges including a talar articular ridge and talar stems with screw fixation holes including three screws placed for additional fixation into the talus according to one or more example implementations of the disclosure;

FIG. 8A is a right view illustrating a talar component with screw flanges and no talar stems according to one or more example implementations of the disclosure;

FIG. 8B is a back view illustrating a talar component with screw flanges and no talar stems according to one or more example implementations of the disclosure;

FIG. 8C is a top view illustrating a talar component with screw flanges and no talar stems according to one or more example implementations of the disclosure;

FIG. 8D is a bottom view illustrating a talar component with screw flanges and no talar stems according to one or more example implementations of the disclosure;

FIG. 8E is a right view from medial of a human right lower extremity and ankle following placement of a reverse ankle arthroplasty system including the tibial component, polyethylene component, and talar component with screw flanges and no talar stems with screw fixation holes including three screws placed for additional fixation into the talus according to one or more example implementations of the disclosure; and

FIG. 8F is a back view of a human right lower extremity and ankle following placement of a reverse ankle arthroplasty system including the tibial component, polyethylene component, and talar component with screw flanges and without talar stems with screw fixation holes including three screws placed for additional fixation into the talus according to one or more example implementations of the disclosure.

Like reference symbols in the various drawings indicate like elements.

### DETAILED DESCRIPTION

Implants may be used, e.g., when one or more human (or animal) body parts need replacing. For example, known ankle replacement implants may have been developed, in order to treat such problems as, e.g., degenerative conditions of the ankle joint including ankle osteoarthritis. Ankle replacement systems often include components which provide two or more bearing surfaces articulate with each other in order to provide prosthetic articulation to allow capability for improved function of the ankle joint. However, such implants of the prior art may be associated with complications, especially, aseptic loosening, which may then require secondary revision surgical procedures.

As will be discussed in greater detail below, the example reverse ankle replacement system may, in some implementations, be able to increase the planar undersurface area of the talar component and, thereby, increase the area for osseous-integration with underlying bone. The example reverse ankle replacement system may also reduce the distance of the axis of rotation from the polyurethane component to the bottom of the undersurface of the talar component which may increase the stability of the talar component. This can lead to decreases in incidence of aseptic loosening in ankle replacement procedures as relates to the reverse ankle replacement system of the present disclosure.

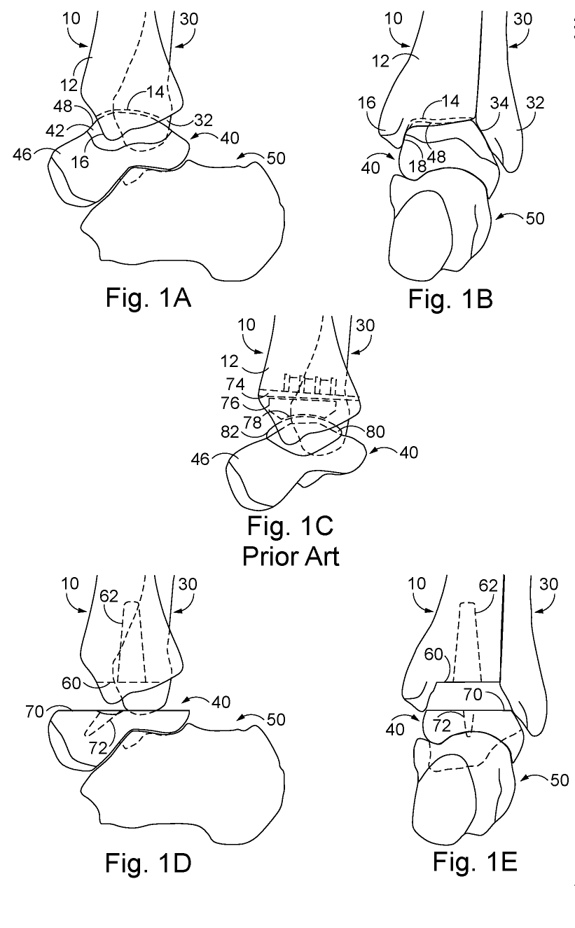

FIG. 1A is a right view from medial of a human right lower extremity and ankle including multiple osseous landmarks with focus on the bony anatomy of the ankle joint. A tibia 10 extends distally, forming the distal tibia 12 and contributes an osseous constraint for the talus, which includes the tibial plafond articular surface 14. The fibula 30 also extends downward forming the lateral malleolus 32 which provides a second osseous constraint for the talus. The talus 40 includes a talar dome 42 along its superior surface, talar head 46 along its anterior aspect, and talar dome articular surface 48 located along the talar dome.

The calcaneus 50 provides a foundation for weight-bearing and articulates with the inferior aspect of the talus. FIG. 1B is a back view from posterior illustrating a human right lower extremity and ankle including a distal tibia, distal fibula, talus, and calcaneus. The distal tibia 12 includes the medial malleolus 16 and medial malleolar articular 18, and the fibula 30 includes its distal aspect, the lateral malleolus 32, and lateral malleolar articular surface 34. Bony constraints of the talus 40 include the medial malleolus and lateral malleolus. FIG. 1C is a right view from medial of a human right lower extremity and ankle including tibia 10, fibula 30, and talus 40, following an ankle replacement procedure, according to a previous device. Tibial component 74 has been implanted into the prepared distal tibia 12, along with placement of intermediate component 76 which has intermediate component bearing undersurface 78 which is concave in shape. The talus has also received talar component 80 which includes talar component bearing surface 82 which is convex in and mates to the concave intermediate component undersurface forming an articulation. The FIG. 1D is a right view of a human right lower extremity and ankle demonstrating tibia and talus bone osteotomies and preparation for placement of reverse ankle replacement prostheses, for at least one embodiment of the present invention. A transverse tibial osteotomy 60 has been performed and a tibial stem channel 62 has been drilled into the distal tibia from distal to proximal, aided with surgical caliper and surgical rulers and drill guides and cone-shaped drill bits, as known in the art. A transverse talar osteotomy 70 has also been performed with drilling talar stem channel 72, aided with surgical caliper and surgical rulers and drill guides and cone-shaped drill bits, as known in the art. FIG. 1E is a back view of a human right lower extremity and ankle further demonstrating tibia and talus bone osteotomies and preparation for placement of reverse ankle replacement prostheses. Talar stem channel 72, is drilled in preparation for implantation of a prosthetic talar component.

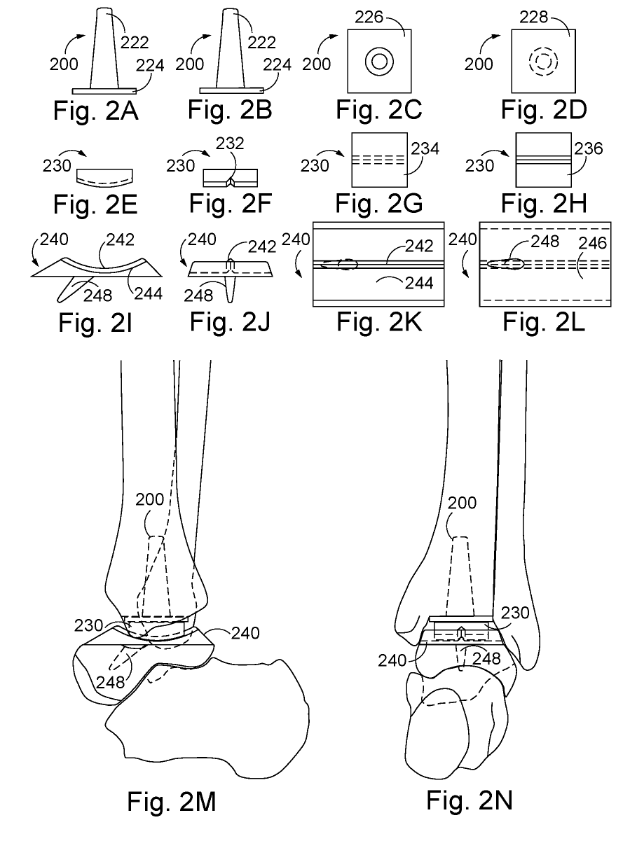

FIG. 2A is a right view illustrating various aspects of a tibial component 200 including a tibial stem 222 and joined tibial articular segment 224, in accordance with one exemplary embodiment of the present invention. The tibial component is inserted into the osteotomized and shaped distal tibial after drilling of the tibial stem channel. FIG. 2B is a back view further illustrating the tibial component and the planar undersurface. FIG. 2C is a top view illustrating the tibial component including a tibial articular segment superior surface 226 and FIG. 2D is a bottom view illustrating a tibial articular segment inferior surface 228 with its smooth planar undersurface. The tibial articular segment inferior surface is highly polished and provides a smooth bearing surface for sliding articulation. The top of the tibial component, tibial articular segment superior surface 226, may include a porous surface to allow for bony ingrowth, along with application of a porous surface to the tibial stem. FIGS. 2E and 2F are a right view and back view, respectively, illustrating a polyethylene component 230 including a polyethylene component recess 232, in accordance with one exemplary embodiment of the present invention. FIG. 2G is a top view illustrating the polyethylene component and polyethylene component superior articular surface 234. The polyethylene component 240 articulates with tibial articular segment inferior surface 228 when the components are implanted and slides along the polished undersurface of the talar component providing rotational sliding capability. FIG. 2H is a bottom view the polyethylene component including a polyethylene component inferior articular surface 236. Both the polyethylene component superior articular surface and polyethylene component inferior articular surface are highly polished and provide smooth bearing surfaces for articulation. FIG. 2I is a right view illustrating a talar component 240 including a talar articular ridge 242 and a talar component articular surface 244, in accordance with one exemplary embodiment of the present invention Both the talar component articular surface and talar articular ridge are concave, and are joined together.

The talar articular ridge is helpful in keeping the polyethylene component retained centrally, along its prominence. FIG. 2J is a back view illustrating the talar component including the talar articular ridge 242 and joined talar stem 248. The polyethylene component 230 articulates with talar component 240 with the talar articular ridge 242 mated and articulating along polyethylene component recess 232 when the components are implanted and during functional activity. FIG. 2K is a top view illustrating the talar component including the talar articular ridge and talar component articular surface 244 and FIG. 2L is a bottom view further illustrating the talar stem 248 and talar component undersurface 246. The talar component articular surface and talar articular ridge are highly polished and provide smooth bearing surfaces for articulation. The talar component undersurface may include a porous surface to allow a wide flat area for bony ingrowth, along with application of a porous surface to the talar stem. The reverse nature of the present talar component and the associated mating articular surface of the polyethylene component is specific to the prosthetic bearing surfaces. The native morphology of the human talar dome is convex in the sagittal plane. The bearing surface of the talar component of the present invention is the reversed, whereby the prosthetic talar component includes an articular surface which is concave as viewed from its left and right sides and the mating articular undersurface of the associated polyethylene component is convex, inferiorly, as viewed in its left and right sides. By reversing the articular surface, the talar component and utilizes a concave surface which yields a biomechanical advantage. The axis of rotation within talar component is close to the bottom surface of talar component. This allows for a short distance for stress transmission from the axis of rotation of the polyurethane component within the talar component, to the bottom surface of the talar component.

FIG. 2M is a right view from medial and FIG. 2N is a back view from posterior of a human right lower extremity and ankle following implantation of a reverse ankle arthroplasty system including the tibial component 200, polyethylene component 230, and talar component 240, in accordance with one exemplary embodiment of the present invention. The steps for implantation are as illustrated and described in FIGS. 1C to 1D, and as described below. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. The transverse tibial and talar osteotomies are completed, as known in the art. Any intervening soft tissue attachments can be freed using a scalpel blade, if needed, to allow osteotomized joint surface bone tissue to be removed. Drilling has also been performed creating a tibial stem channel, in preparation for implant accommodation of tibial stem 222 of the tibial component. The tibial component and talar component may be applied either with or without the use of bone cement, with choice dependent on surgeon preference. Following implantation of the tibial component and talar component, followed by the polyethylene component, articulation is afforded by the rotational articulation between the tibial articular segment and polyethylene component superior articular surface, thus facilitating internal and external rotation of the ankle joint. Additionally, the articulation between the polyethylene component inferior surface and talar component articular surface allows controlled flexion and extension through a range of motion while the polyethylene component is further retained and controlled through the interface of the polyethylene component recess and talar articular ridge. When properly implanted, the apex of the talar articular ridge is seated into and along the polyethylene component recess.

The tibial component, talar component, and polyethylene component are designed to come in multiple sizing options to enable the surgeon to select appropriate size implants intraoperatively for each individual patient, as needed. Thus, patients with larger anatomy can receive a proportionally larger tibial component, talar component and polyethylene component.

The tibial component and talar component in this embodiment may be manufactured to consist of metal or metal alloy. The polyethylene component may be manufactured to consist of standard polyethylene or highly-crosslinked polyethylene as known in the art, or another suitable polymer, ceramic, or metal or metal alloy or material combinations. The tibial component and talar component can consist of high carbon cobalt chrome (CoCr) alloy. They may also be made of an alternate metal or metal alloy including stainless steel, titanium, or another suitable material including ceramics or combination materials. It can be formed as cast, forged, milled or drilled through machining processes or a combination of methods. Standard machining techniques can be used during component manufacture including for creation of the tapered stems of the tibial component and talar component, and the talar articular ridge. The tibial articular segment superior surface, tibial stem, talar component undersurface, and talar stem, can have a surface to induce osseous-integration along the implant-bone interface. This surface can consist of a porous, textured, granular and/or beaded surface coating as known in the art. The polyethylene component may be manufactured by forming the polyethylene segment as cast or other techniques including injection molding or plastic welding as known in the art. The tibial articular segment inferior surface 228, polyethylene component superior articular surface 234, polyethylene component inferior articular surface 236, talar component articular surface 244 and talar articular ridge 242 and additional surfaces many be further smoothed to achieve a highly-polished finish during production through standard manufacturing techniques, as known in the art.

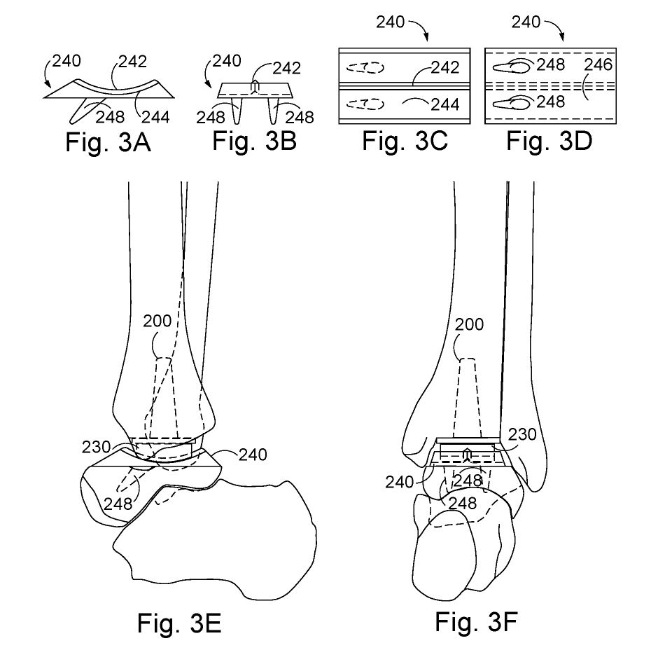

FIG. 3A is a right view illustrating a talar component 240 including talar articular ridge 242 and talar component articular surface 244, in accordance with one exemplary embodiment of the present invention. Both the talar component articular surface and talar articular ridge are concave, and are joined together. The prosthesis and steps for implantation are the same as that illustrated and described in FIGS. 2A to 2N with a few differences, which are described below. FIG. 3B is a back view illustrating the talar component including the talar articular ridge 242 and two of talar stem 248. FIG. 3C is a top view illustrating the talar component including the talar articular ridge and talar component articular surface 244 and FIG. 3D is a bottom view further illustrating two of talar stem 248 and talar component undersurface 246. The two talar stems of this embodiment are included to offer additional stability when seated to the underlying bone of the talus. FIG. 3E is a right view from medial and FIG. 3F is a back view from posterior of a human right lower extremity and ankle following implantation of a reverse ankle replacement system including the tibial component 200, polyethylene component 230, and talar component 240, in accordance with one exemplary embodiment of the present invention. The steps for implantation are as illustrated and described in FIGS. 2A to 2N, with a few differences described below. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. The transverse tibial and talar osteotomies have been completed. Drilling has also been performed, creating tibial stem channel 62, in preparation for implant accommodation of tibial stem 222 of the tibial component. Two of talar stem channels are drilled for fitment of the talar component, corresponding with the two of talar stem 248 of the present embodiment, using surgical caliper, and drill guide, as known in the art. The talar component may be applied either with or without the use of bone cement, with choice dependent on surgeon preference. An advantage of inclusion of two talar stems in this embodiment is that this may afford additional fixation stability in the presence of bone deficiencies, including osteoporotic bone or avascular necrosis.

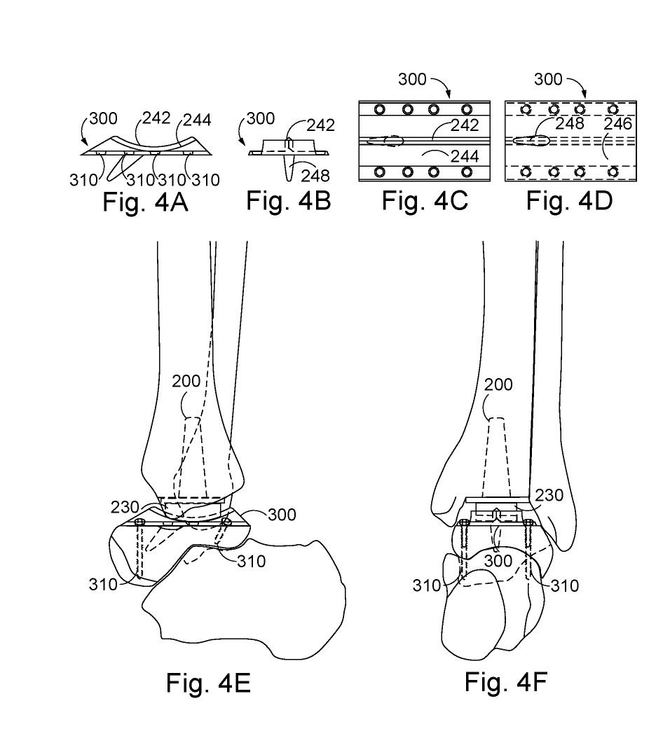

FIG. 4A is a right view illustrating a talar component with screw fixation holes 300 including talar articular ridge 242 and talar component articular surface 244, in accordance with one exemplary embodiment of the present invention. Both the talar component articular surface and talar articular ridge are concave, and are joined together. The prosthesis and steps for implantation are the same as that illustrated and described in FIGS. 2A to 2N with a few differences, which are described below. Four of talar component screw hole 302 are present along the right side of the talar component in this embodiment and an additional four located along the left side. Non-locking screw holes are preferred in this embodiment. However, these screw holes may be either a locking design or non-locking design or include various both locking and non-locking screw holes, as known in the art. The locking screw holes have threads along the periphery of the screw hole to allow the bone screw to engage the threads and effectively lock the screw into the talar component. Non-locking screw holes do not include screw threads along their periphery and thereby facilitate further compressive force upon screw tightening. FIG. 4B is a back view illustrating the talar component including the talar articular ridge 242 and joined talar stem 248. FIG. 4C is a top view further illustrating the talar component including the talar articular ridge and talar component screw holes and talar component articular surface 244 and FIG. 4D is a bottom view further illustrating the talar stem 248 and talar component undersurface 246.

FIG. 4E is a right view from medial and FIG. 4F is a back view from posterior of a human right lower extremity and ankle following implantation of a reverse ankle arthroplasty system including the tibial component 200, polyethylene component 230, and talar component with screw fixation holes 300, in accordance with one exemplary embodiment of the present invention. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. In addition to the surgical steps detailed in FIGS. 2A to 2N, four of bone screw 310 are inserted through the talar component screw holes into the talus to provide additional fixation for the talar component into the underlying cancellous bone of the talus. In the setting of cement-less application, when desired, the addition of these bone screws add additional compression along the bone and implant interface and may improve the stability capability for osseous-integration with porous undersurfaces by further reducing micro-motion along this interface. These screws can be drilled and inserted in standard fashion, and aided with use of a drill depth gauge, surgical caliper, and surgical drill, standard bone screws and instruments, as known in the art.

The talar component with screw fixation holes and bone screws of this embodiment may be manufactured to consist of metal or metal alloy. The talar component can consist of high carbon cobalt chrome (CoCr) alloy. They may also be made of an alternate metal or metal alloy including stainless steel, titanium, or another suitable material. It can be formed as cast, forged, milled or drilled through machining processes or a combination of methods. Standard machining techniques can be used during component manufacture including for creation of the tapered stem of the talar component, and the talar articular ridge. The talar component undersurface, and talar stems, can have a surface to induce osseous-integration along the implant-bone interface. This surface can consist of a porous, textured, granular and/or beaded surface coating as known in the art. The articular surfaces and additional surfaces many be further smoothed during production, and standard manufacturing techniques as known in the art. The bearing articular surfaces are also highly polished during the manufacturing process through standard machining processes.

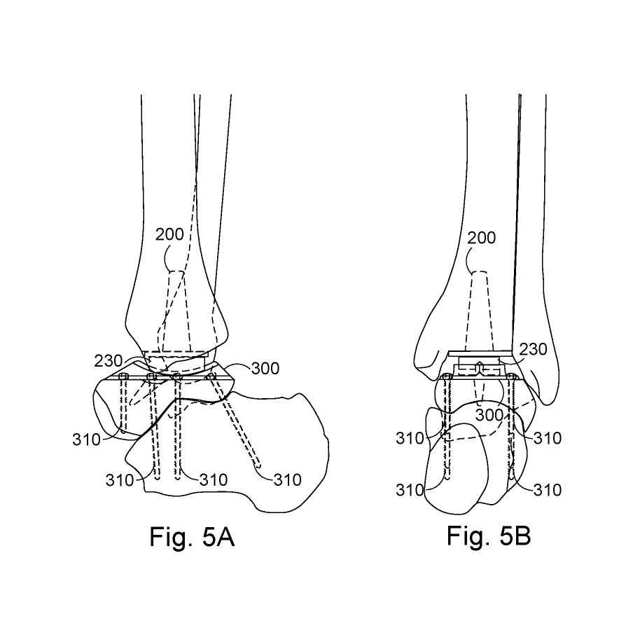

FIG. 5A is a right view from medial and FIG. 5B is a back view from posterior of a human right lower extremity and ankle following implantation of a reverse ankle arthroplasty system including the tibial component 200, polyethylene component 230, and talar component with screw fixation holes 300, in accordance with one exemplary embodiment of the present invention. The prosthesis and steps for implantation are the same as that illustrated and described in FIGS. 4A to 4F with a few differences, which are described below. In addition to the surgical steps detailed in FIGS. 4A to 4F, a total of eight of bone screw 310 are inserted and secured through the talar component screw holes with the two anterior screws into the talus and the remaining screws through the talus and into the calcaneus to provide additional fixation for the talar component into the underlying cancellous bone of the talus while also fusing the sub-talar joint. In the setting of cement-less application, when desired, the addition of these bone screws adds additional compression along the bone and implant interface and may improve the ability for osseous-integration with porous undersurfaces by further reducing excessive micro-motion along this interface. Non-locking screw holes are preferred in this embodiment. However, these screw holes may be either a locking design or non-locking design or include various both locking and non-locking screw holes, as known in the art. The locking screw holes have threads along the periphery of the screw hole to allow the bone screw to engage the threads and effectively lock the screw into the talar component. Non-locking screw holes do not include screw threads along their periphery and thereby facilitate further compressive force upon screw tightening. The screws which cross the sub-talar joint fuse this joint in order to provide pain relief in the setting of advanced sub-talar joint arthritis. An advantage of placement of these screws through the prosthesis is that the fixation is connected with talus component and afforded additional stability as the screws are all connected to the same implant. These screws can be drilled and inserted in standard fashion, and aided with use of a drill depth gauge, surgical caliper, and surgical drill, standard bone screws and instruments, as known in the art.

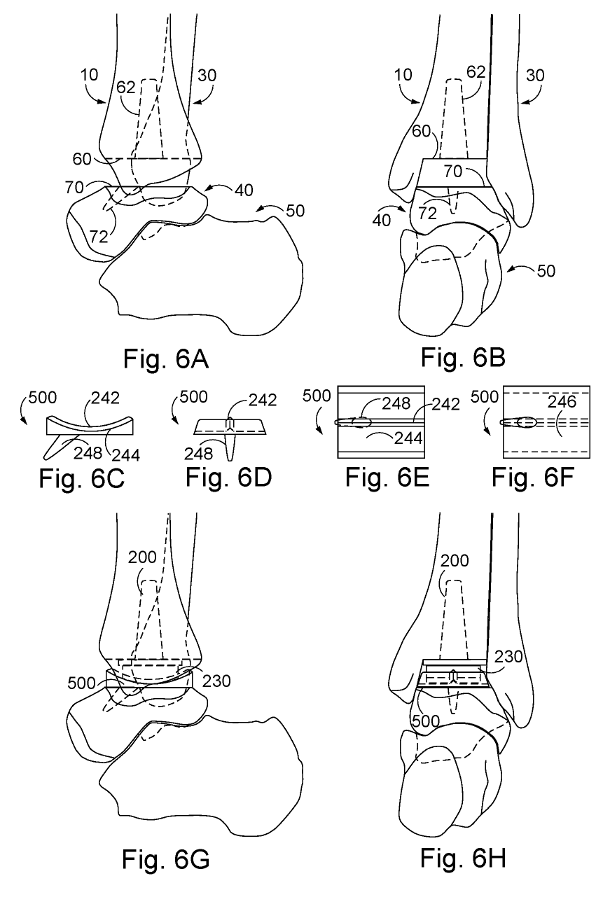

FIG. 6A is a right view of a human right lower extremity and ankle demonstrating tibia and talus bone osteotomies and preparation for placement of an alternate reverse ankle replacement prostheses, for at least one embodiment of the present invention. The prosthesis and steps for implantation are the same as that illustrated and described in FIGS. 2A to 2N with a few differences, which are described below. A transverse tibial osteotomy 60 has been performed more proximally along distal tibia, removing more tibial bone and a tibial stem channel 62 has also been drilled into the distal tibia from distal to proximal, aided with surgical caliper and surgical rulers and drill guides, as known in the art. A transverse talar osteotomy 70 has also been performed more proximally in the talus, removing a reduced amount of talus bone, with drilling talar stem channel 72, aided with surgical caliper and surgical rulers and drill guides, as known in the art. FIG. 6B is a back view of a human right lower extremity and ankle further demonstrating tibia and talus bone osteotomies and preparation for placement of an alternate reverse ankle replacement prostheses. Talar stem channel 72, is drilled in standard fashion, in preparation for implantation of a prosthetic talar component.

FIG. 6C is a right view illustrating a shortened talar component 500 including talar articular ridge 242 and talar component articular surface 244, in accordance with one exemplary embodiment of the present invention. Both the talar component articular surface and talar articular ridge are concave, and are joined together. The prosthesis and steps for implantation are the same as that illustrated and described in FIGS. 2A to 2N with a few differences, which are described below. The overall length of the talar component from its front surface to its back at its lower aspect has been shortened in this embodiment while the length of the talar component articular surface remains the same as the previous embodiments. FIG. 6D is a back view illustrating the shortened talar component 500 including the talar articular ridge 242 and joined talar stem 248. FIG. 6E is a top view further illustrating the shortened talar component including the talar articular ridge and talar component articular surface 244, and FIG. 6F is a bottom view further illustrating the talar stem 248 and talar component undersurface 246.

FIG. 6G is a right view from medial and FIG. 6H is a back view from posterior of a human right lower extremity and ankle following implantation of a reverse ankle arthroplasty system including the tibial component 200, polyethylene component 230, and shortened talar component 500, in accordance with one exemplary embodiment of the present invention. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. The shortened talar component of this embodiment allows for a decreased footprint for the talar component along the talus and may help to reduce overall talar bone resection required for surgical implantation. This reduced bone resection volume may be beneficial when improved bone quality is encountered and it is desired to retain more of the native talus for implantation of a talar component.

The shortened talar component in this embodiment may be manufactured to consist of metal or metal alloy. The talar component can consist of high carbon cobalt chrome (CoCr) alloy. They may also be made of an alternate metal or metal alloy including stainless steel, titanium, or another suitable material. It can be formed as cast, forged, milled or drilled through machining processes or a combination of methods. Standard machining techniques can be used during component manufacture including for creation of the tapered stem of the talar component, and the talar articular ridge. The talar component undersurface, and talar stem, can have a surface to induce osseous-integration along the implant-bone interface. This surface can consist of a porous, textured, granular and/or beaded surface coating as known in the art. The articular surfaces and additional surfaces many be further smoothed during production, and standard manufacturing techniques as known in the art. The bearing articular surfaces are also highly polished during the manufacturing process through standard machining processes.

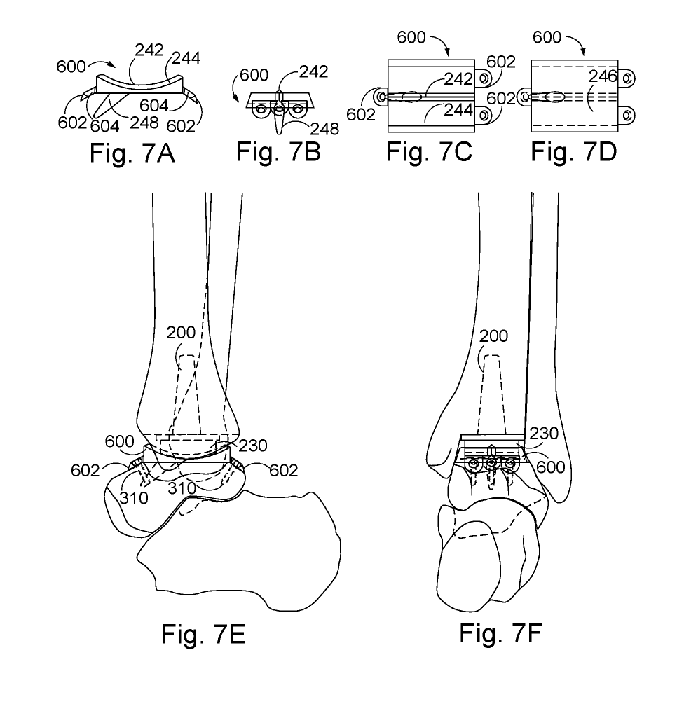

FIG. 7A is a right view and FIG. 7B is a back view illustrating a talar component with screw flanges 600 including three of flange 602, each with flange screw hole 604, also including talar articular ridge 242 and talar component articular surface 244, in accordance with one exemplary embodiment of the present invention. Both the talar component articular surface and talar articular ridge are concave, and are joined together. The prosthesis is the same as that illustrated and described in FIGS. 6A to 6H with a few differences, which are described below. FIG. 7C is a top view further illustrating the talar component including the flanges, talar articular ridge and talar component articular surface 244 and FIG. 7D is a bottom view further illustrating the talar component undersurface 246. One of flange 602 is present along the front aspect of the talar component with screw flanges of this embodiment and two flanges are present along the back aspect. Located within the center of each flange is a flange screw hole 604. Non-locking flange screw holes are preferred in this embodiment. However, these screw holes may be either a locking design or non-locking design or include various both locking and non-locking screw holes, as known in the art. The locking screw holes have threads along the periphery of the screw hole to allow the bone screw to engage the threads and effectively lock the screw into the talar component. Non-locking screw holes do not include screw threads along their periphery and thereby facilitate further compressive force upon screw tightening.

The steps for implantation are the same as that illustrated and described in FIGS. 6A to 6H with a few differences, which are described below. FIG. 7E is a right view from medial and FIG. 7F is a back view from posterior of a human right lower extremity and ankle following implantation of a reverse ankle arthroplasty system including the tibial component 200, polyethylene component 230, and talar component with screw flanges 600 with use of 3 of bone screw 310, in accordance with one exemplary embodiment of the present invention. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. In addition to the surgical steps detailed in FIGS. 6A to 6H, three of bone screw 310 are inserted and secured through the flange screw holes 604 of each of the three flange 602 and into the talus to provide additional fixation for the talar component into the underlying cancellous bone of the talus. The two flanges at the back of the talar component in this embodiment are situated to be seated along the back-left and back-right aspect of the talus in order to allow for easier access during surgical exposure as the Achilles tendon can be avoided during retraction. In the setting of cement-less application, when desired, the addition of these bone screws adds additional compression along the bone and implant interface and may improve the ability for osseous-integration with porous undersurfaces by further reducing micro-motion along this interface. These screws can be drilled and inserted in standard fashion, and aided with use of a drill depth gauge, surgical caliper, and surgical drill, standard bone screws and instruments, as known in the art.

The talar component with screw flanges in this embodiment may be manufactured to consist of metal or metal alloy. The talar component can consist of high carbon cobalt chrome (CoCr) alloy. They may also be made of an alternate metal or metal alloy including stainless steel, titanium, or another suitable material. It can be formed as cast, forged, milled or drilled through machining processes or a combination of methods. Standard machining techniques can be used during component manufacture including for creation of the tapered stems of the talar component, and the talar articular ridge. The talar component undersurface, and talar stems, can have a surface to induce osseous-integration along the implant-bone interface. This surface can consist of a porous, textured, granular and/or beaded surface coating as known in the art. The articular surfaces and additional surfaces many be further smoothed during production, and standard manufacturing techniques as known in the art. The bearing articular surfaces are also highly polished during the manufacturing process through standard machining processes.

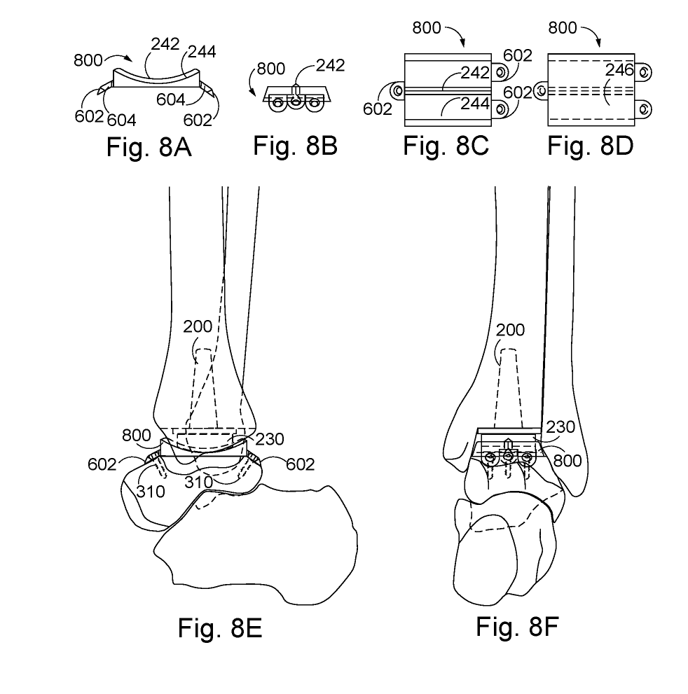

FIG. 8A is a right view and FIG. 8B is a back view illustrating a talar component with screw flanges and no talar stems 800 including three of flange 602, each with flange screw hole 604, and including talar articular ridge 242 and talar component articular surface 244, in accordance with one exemplary embodiment of the present invention. Both the talar component articular surface and talar articular ridge are concave, and are joined together. The prosthesis and steps for implantation are the same as that illustrated and described in FIGS. 7A to 7F with a few differences, which are described below. The talar stem has been omitted in this embodiment to ease implantation and further reduce requisite bone resection for surgical implantation. This further reduction in bone resection may be preferred in the setting of excellent bone quality in the talus further minimizing bone removal during the surgical procedure. FIG. 8C is a top view further illustrating the talar component including the flanges, talar articular ridge and talar component articular surface 244 and FIG. 8D is a bottom view further illustrating the talar component undersurface 246.

FIG. 8E is a right view from medial and FIG. 8F is a back view from posterior of a human right lower extremity and ankle following implantation of a reverse ankle arthroplasty system including the tibial component 200, polyethylene component 230, and talar component with screw flanges and no talar stems with use of 3 of bone screw 310, in accordance with one exemplary embodiment of the present invention. Either posterior, anterior or combined surgical approaches or may be utilized for access to and arthrotomy of the ankle joint for performing the surgical steps including arthroplasty. An optional fibular osteotomy may also be performed to further aid surgical exposure and ankle joint access and visualization of the joint surfaces through a peripheral lateral surgical approach. In contrast to the talus component embodiment of FIGS. 7A to 7F, a talar stem channel is not drilled into the talus to accommodate a talar stem as the talar stem is omitted in this embodiment. As in the surgical steps detailed in FIGS. 7A to 7F, three of bone screw 310 are inserted and secured through the flange screw holes of each of the three flange 602 component screw holes into the talus to provide additional fixation for the talar component into the underlying cancellous bone of the talus. In the setting of cement-less application, when desired, the addition of these bone screws adds additional compression along the bone and implant interface and may improve the ability for osseous-integration with porous undersurfaces by further reducing micro-motion along this interface. These screws can be drilled and inserted in standard fashion, and aided with use of a drill depth gauge, surgical caliper, and surgical drill, standard bone screws and instruments, as known in the art. The omission of talar stems in this embodiment avoids the additional associated bone resection which would be required for talar stems and can help preserve additional talar bone during procedure.

The talar component with screw flanges and no talar stems in this embodiment may be manufactured to consist of metal or metal alloy. The talar component can consist of high carbon cobalt chrome (CoCr) alloy. They may also be made of an alternate metal or metal alloy including stainless steel, titanium, or another suitable material. It can be formed as cast, forged, milled or drilled through machining processes or a combination of methods. Standard machining techniques can be used during component manufacture including for creation of the talar articular ridge. The talar component undersurface can have a surface to induce osseous-integration along the implant-bone interface. This surface can consist of a porous, textured, granular and/or beaded surface coating as known in the art. The articular surfaces and additional surfaces many be further smoothed during production, and standard manufacturing techniques as known in the art. The bearing articular surfaces are also highly polished during the manufacturing process through standard machining processes.

The tibial components and talar components of the present invention provide means for both un-cemented and cemented application and options for multiple fixation modes. They are intended to be implanted along with the polyethylene component to effectively replace the native ankle joint in the setting of advanced ankle joint arthritis. Depending on underlying bone quality within the talus an embodiment can be selected to minimize bone resection or provide capability for supplemental screw fixation in talar component embodiments with screw holes. In multiple exemplary embodiments, additional construct support and compression along the talar component and bone interface is achievable through use of supplemental bone screw fixation through flanges screw holes when needed. Sub-talar fusion is possible directly through the talar component by using longer bone screws which pass through the talus and into the calcaneus. Through selection of various exemplary embodiments, construct support options can be chosen based upon individualized need for fixation and stability also allowing the surgeon to address bone deficiencies as well as sub-talar joint arthritis.

While the above description contains much specificity, this should not be construed as limitations on the scope, but rather an exemplification of one or more exemplary embodiments as detailed. Multiple additional configurations are possible of the tibial component, polyethylene component and talar components. Inclusion of a different number of flanges and or screw holes in the described embodiments is possible with, but not limited to one-, two- or four-flanges or different combinations of screw hole in any embodiment. Inclusion of a greater number of talar stems is possible for all embodiments, for example two or three talar stems may be utilized in any embodiment. The talar stems may also be omitted from any embodiment. The talar stems may also be joined at an altered angular direction from the undersurface of the talar component undersurface. The talar articular ridge may be omitted but its presence is preferred to aid in the localization of the polyethylene component along the bearing surface of the talar component.

Additionally, the flanges illustrated in FIGS. 7A to 7F and FIGS. 8A to 8F may instead be located and in duplicate along the periphery of the talar component along the front-left and front-right and additionally a single flange present along the back-center of the talar component instead of in duplicate as illustrated. The flanges may also be joined to the talar component at an altered angle. Additionally, the flange screw hole may be omitted from any flange, but is presently preferred for inclusion as it provides variable screw fixation capabilities. A different number of talar component screw holes may be present or locations altered in the talar component with screw fixation hole embodiment illustrated in FIGS. 4A to 4F and FIGS. 5A to 5B. These alterations in screw holes would achieve corresponding alternate screw locations into the underlying talar bone when implanted.

The tibial component and polyethylene component may be joined together, during manufacture, effectively reducing the reverse ankle replacement system from the present three-piece component design to a two-piece component design. However, the three-piece component design in the present disclosure is preferred, as rotational ability is optimized through the sliding capability of the polyethylene component along the undersurface of the tibial component, rather then a fixed join between these two components. The tibial stem may also be shortened in vertical height and/or angled to ease bone preparation and implantation.

Additionally, the talar component and associated polyethylene component may be manufactured with a higher or lower degree of curvature. Increasing the degree of curvature may promote the polyethylene retaining capability in the setting of increased ankle joint laxity. The edges of the articular bearing surfaces and other edges may be filleted or beveled during manufacturing for the tibial component, polyethylene component and talar component.

The height of the talar and tibial osteotomies may be changed with corresponding changes made to the height of the tibial and talar components to adjust the amount of bone resection requisite for surgical implantation. The width of the talar component may also be altered to adjust for resection height. Finally, hollowing out of the talar component undersurface can allow additional native talar bone to be preserved but the illustrated embodiments are preferred to optimize ease of implantation using a transverse osteotomy of the talus.

In all talar component and tibial component embodiments, a porous texture along the undersurface and stem aspects, which interfaces with the native bone, may be omitted, but the porous texture is however preferred to allow osseous-integration with the implant. Bone screws may be removed after osseous-integration has been achieved in talar component embodiments where bone screw fixation options are provided. However, it is currently preferred to retain bone screws to avoid a secondary surgical procedure for screw removal.

The tibial component, polyethylene component, and talar component, and bone screws, may each be made of metal or metal alloy including high carbon cobalt chrome (CoCr) alloy, stainless steel, titanium or aluminum, ceramic-coated metal, oxidized metals, polyethylene, or another suitable material, or combination of suitable materials. They may be manufactured as cast or using additional standard techniques including injection molding, forging, bending during the machining process, and can be manufactured in segments and welded, plastic welded or otherwise joined, e.g., with an inference fit.

Although the present invention has been described with reference to particular examples and exemplary embodiments, it should be understood that the foregoing description is in no manner limiting. For example, although the present invention has been described with reference to particular materials, manufacturing methods and joining methods, it should be understood that other suitable materials such as for example plastics or ceramics, other manufacturing methods such as for example injection molding, laser cutting or alternate machining methods, and other joining methods such as for example friction fitting may be encompassed by the present invention. Moreover, the features described herein may be used in any combination.

While one or more example implementations have been described using a reverse ankle replacement prosthesis for a human, it will be appreciated that the present disclosure may be adapted for use for animals as well. As such, the description of a reverse ankle replacement prosthesis for a human should be used as example only and not to otherwise limit the scope of the disclosure.

What is claimed is:

1. A reverse ankle arthroplasty prosthesis comprising:
a talar component, wherein the talar component includes a talar component articular surface which is concave and configured to provide a smooth bearing surface for articulation.

2. A reverse ankle arthroplasty prosthesis comprising:
a polyethylene component, wherein the polyethylene component includes a inferior articular surface which is convex and configured to provide a smooth bearing surface for articulation.

3. The reverse ankle arthroplasty prosthesis according to claim 1 further comprising at least one talar component screw hole.

4. The reverse ankle arthroplasty prosthesis according to claim 1 further comprising at least one flange.

5. The reverse ankle arthroplasty prosthesis according to claim 1 further comprising at least one porous surface configured to promote bony ingrowth.

6. A reverse ankle arthroplasty prosthesis comprising:
a tibial component sized and arranged to attach to the tibia, wherein the tibial component includes a planar undersurface;
a talar component sized and arranged to attach to the talus, wherein the talar component includes a bearing surface which is concave; and
a polyethylene component sized and arranged to be placed between the tibial component and talar component, wherein the polyethylene component includes a bearing undersurface which is convex.

7. The reverse ankle arthroplasty prosthesis according to claim 6 wherein the tibial component includes a surface that is at least one of porous, textured, granular, and beaded.

8. The reverse ankle arthroplasty prosthesis according to claim 6 wherein the talar component includes a surface that is at least one of porous, textured, granular, and beaded.
